(19) United States
(12) Patent Application Publication
(10) Pub. No.: US 2019/0247350 A1
(43) Pub. Date: Aug. 15, 2019

(54) COMPOSITION FOR IMPROVING DECREASED ABSORPTION IN DIGESTIVE TRACT, AND COMPOSITION FOR PROMOTING ABSORPTION IN DIGESTIVE TRACT

(71) Applicant: AJINOMOTO CO., INC., Tokyo (JP)

(72) Inventors: Ami MIZUGAKI, Kawasaki-shi (JP); Sakiko TOYODA, Kawasaki-shi (JP); Yoshihito NOGUSA, Kawasaki-shi (JP)

(73) Assignee: AJINOMOTO CO., INC., Tokyo (JP)

(21) Appl. No.: 16/394,431

(22) Filed: Apr. 25, 2019

Related U.S. Application Data
(63) Continuation of application No. PCT/JP2017/038423, filed on Oct. 25, 2017.

(30) Foreign Application Priority Data
Oct. 26, 2016 (JP) 2016-209926

(51) Int. Cl. A61K 31/198; A61P 1/14; A23L 33/175

(52) U.S. Cl. CPC A61K 31/198; A61P 1/14; A23L 33/175; A23V 2200/32; A23V 2250/0616; A23V 2250/062; A23V 2002/00

(57) ABSTRACT

Compositions which contain at least one of cystine and glutamine as an active ingredient are useful for improving decreased absorption in the gastrointestinal tract or promoting absorption in the gastrointestinal tract.

### CROSS REFERENCES TO RELATED APPLICATIONS

This application is a continuation of International Patent Application No. PCT/JP2017/038423, filed on Oct. 25, 2017, and claims priority to Japanese Patent Application No. 2016-209926, filed on Oct. 26, 2016, both of which are incorporated herein by reference in their entireties.

### BACKGROUND OF THE INVENTION

### Field of the Invention

The present invention relates to compositions for improving decreased absorption in the gastrointestinal tract. In addition, the present invention relates to compositions for promoting absorption in the gastrointestinal tract.

### Discussion of the Background

Digestion and absorption of nutrients in the gastrointestinal tract is performed in three steps of (1) hydrolysis of fats, proteins and carbohydrates by intracameral enzymes, (2) digestion with brush border enzymes and uptake of the final products, and (3) lymphatic transport of nutrients, and absorption in the gastrointestinal tract decreases and malabsorption of nutrients occurs when any of these steps is impaired.

Decreased absorption in the gastrointestinal tract is caused by insufficient mixing in the stomach caused by operation of stomach resection by Billroth II method, gastrocolic fistula, gastroenterostomy or the like, rapid discharge; insufficient digestive enzymes due to biliary obstruction, chronic liver failure, chronic pancreatitis, cystic fibrosis, lactase deficiency, pancreatic cancer and the like; exacerbation of the gastrointestinal-tract environment due to diabetes, scleroderma, abnormal mobility secondary to hyperthyroidism, abnormal proliferation of enteric bacterium and the like; acute abnormality of mucosal epithelium due to acute infectious intestinal diseases, alcohol, neomycin and the like; chronic abnormality of mucosal epithelium due to amyloidosis, Celiac disease, Crohn's disease and the like; transport disorder due to abeta-lipoproteinemia, Addison's disease, lacteal obstruct lymphoma, tuberculosis, lymphangiectasis and the like; and the like.

The gastrointestinal tract has a system that prevents invasion of harmful foreign substances in the gastrointestinal tract into the body, that is, an intestinal barrier function. It has been reported that various stress loads induce abnormalities in the intestinal barrier function and the resulting invasion of foreign substances causes inflammation in the gastrointestinal tract and various organs and disturbance of the immune system (see Takuya Suzuki; The Uehara Memorial Foundation report 26 1-6 (2012), which is incorporated herein by reference in its entirety).

Furthermore, a decrease in the intestinal barrier function due to intense exercise has been reported (see Kim van Wijck et al.; Plos One 6 (7) e22366 (2011), which is incorporated herein by reference in its entirety).

It is considered that such a decrease in the intestinal barrier function induces dysfunction of the gastrointestinal tract and, as a result, reduces digestion and absorption capacity in the gastrointestinal tract.

When a state of reduced absorption in the gastrointestinal tract persists, substances that remain in the body without being absorbed cause emergence of symptoms of diarrhea, steatorrhea, abdominal swelling, gas and the like. In addition, malabsorption of particular nutrients causes symptoms of anemia, peliosis, petechia, carpopedal spasm, edema, glossitis, nyctalopia, pain in the limbs and bones, pathological bone fracture, peripheral nerve disorders and the like.

Therefore, there is a demand for an effective composition for improving decreased absorption in the gastrointestinal tract induced by various causes.

Particularly, in the modern society, stress is constantly and continuously loaded, and reduction thereof is often difficult. To maintain health of those who cannot avoid exercise such as athletes, it is highly necessary to improve decreased absorption in the gastrointestinal tract caused by stress and exercise.

### SUMMARY OF THE INVENTION

Accordingly, it is one object of the present invention to provide novel compositions for improving decreased absorption in the gastrointestinal tract induced by various causes, particularly, a composition for improvement which is capable of well improving decreased absorption in the gastrointestinal tract induced by stress or exercise

This and other objects, which will become apparent during the following detailed description, have been achieved by the inventors' discovery that at least one of cystine and glutamine can improve decreased absorption in the gastrointestinal tract.

The present inventors have also found that at least one of cystine and glutamine promotes absorption in the gastrointestinal tract.

That is, the present invention relates to the following.

(1) A composition for improving decreased absorption in the gastrointestinal tract, comprising at least one of cystine and glutamine as an active ingredient.

(2) The composition of (1), comprising cystine and glutamine.

(3) The composition of (2), wherein a content ratio of cystine and glutamine (cystine:glutamine) is 1:0.01 to 1:100 in weight ratio.

(4) The composition of any of (1) to (3), which is a composition for improving decreased absorption of water in the gastrointestinal tract.

(5) The composition of any of (1) to (3), which is a composition for improving decreased absorption of nutrient in the gastrointestinal tract.

(6) The composition of (5), wherein the nutrient is at least one selected from the group consisting of protein, peptide, amino acid, carbohydrate, lipid, vitamin and mineral.

(7) The composition of (6), wherein the vitamin is at least one selected from the group consisting of vitamin A, vitamin B group, vitamin D and vitamin E.

(8) The composition of any of (1) to (7), which is a pharmaceutical composition.

(9) The composition of any of (1) to (7), which is a food composition.

(10) A method for improving decreased absorption in the gastrointestinal tract, comprising ingestion by or administration to a target showing decreased absorption in the gastrointestinal tract of at least one of cystine and glutamine in an amount effective for improving the decreased absorption in the gastrointestinal tract.

(11) The method of (10), comprising ingestion or administration of cystine and glutamine in amounts effective for improving the decreased absorption in the gastrointestinal tract.

(12) The method of (11), wherein a content ratio of the cystine and glutamine (cystine:glutamine) is 1:0.01 to 1:100 in a weight ratio.

(13) The method of any of (10) to (12), which improves decreased absorption of water in the gastrointestinal tract.

(14) The method of any of (10) to (12), which improves decreased absorption of nutrient in the gastrointestinal tract.

(15) The method of (14), wherein the nutrient is at least one selected from the group consisting of protein, peptide, amino acid, carbohydrate, lipid, vitamin and mineral.

(16) The method of (15), wherein the vitamin is at least one selected from the group consisting of vitamin A, vitamin B group, vitamin D and vitamin E.

(17) A composition for promoting absorption in the gastrointestinal tract, comprising at least one of cystine and glutamine as an active ingredient.

(18) The composition of (17), which is a composition for promoting absorption of nutrient in the gastrointestinal tract.

(19) The composition of (18), wherein the nutrient is at least one selected from the group consisting of protein, peptide, amino acid, carbohydrate, lipid, vitamin and mineral.

(20) The composition of any of (17) to (19), which is a pharmaceutical composition.

(21) The composition of any of (17) to (19), which is a food composition.

(22) A method for promoting absorption in the gastrointestinal tract, comprising ingestion by or administration to a target in need of promotion of absorption in the gastrointestinal tract of at least one of cystine and glutamine in an amount effective for promoting absorption in the gastrointestinal tract.

(23) The method of (22), which promotes absorption of nutrient in the gastrointestinal tract.

(24) The method of (23), wherein the nutrient is at least one selected from the group consisting of protein, peptide, amino acid, carbohydrate, lipid, vitamin and mineral.

### Effect of the Invention

The composition of the present invention for improving decreased absorption in the gastrointestinal tract can improve decreased absorption in the gastrointestinal tract, which is induced by various causes, particularly, stress or exercise.

That is, the composition of the present invention for improving decreased absorption in the gastrointestinal tract can suppress decreased absorption of water, nutrient and the like via the gastrointestinal tract due to some cause such as stress, exercise and the like, and can improve absorption of water, nutrients and the like via the gastrointestinal tract from a decreased state due to some cause to a normal state or a good state.

Furthermore, the composition of the present invention for promoting absorption in the gastrointestinal tract can promote absorption of nutrients and the like via the gastrointestinal tract.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 shows the effect of exercise and cystine on the expression of peptide transporter gene in Experimental Example 1. In the Figure, “s” indicates that a difference tending to be significant is found at p<0.1, and “**” indicates that it is significant at p<0.01.

FIG. 2 shows the effect of exercise and glutamine on the expression of amino acid transporter gene in Experimental Example 1. In the Figure, “*” indicates that it is significant at P<0.05.

FIG. 3 shows the effect of exercise and glutamine on the expression of scavenge receptor class B gene in Experimental Example 1. In the Figure, “s” indicates that a difference tending to be significant is found at p<0.1.

FIG. 4 shows the effect of exercise and glutamine on the expression of ABC protein G8 gene in Experimental Example 1. In the Figure, “*” indicates that it is significant at P<0.05, and “**” indicates that it is significant at p<0.01.

FIG. 5 shows the effect of exercise and cystine on the expression of ABC protein G5 gene in Experimental Example 1. In the Figure, “*” indicates that it is significant at P<0.05, and “**” indicates that it is significant at p<0.01.

FIG. 6 shows the effect of exercise and glutamine on the expression of folic acid transporter gene in Experimental Example 1. In the Figure, “*” indicates that it is significant at P<0.05.

FIG. 7 shows the effect of exercise and cystine on the expression of biotinidase gene in Experimental Example 1. In the Figure, “**” indicates that it is significant at p<0.01.

FIG. 8 shows the effect of exercise and cystine on the expression of sodium-dependent multivitamin transporter gene in Experimental Example 1. In the Figure, “*” indicates that it is significant at P<0.05, and “**” indicates that it is significant at p<0.01.

FIG. 9 shows the effect of exercise, glutamine and cystine on the expression of aquaporin gene in Experimental Example 1. In the Figure, “**” indicates that it is significant at p<0.01.

FIG. 10 shows the effect of exercise and cystine on the glucose absorption capacity in Experimental Example 2. In the Figure, “*” indicates that it is significant at P<0.05.

FIG. 11 shows the effect of exercise and cystine on the expression of sodium-dependent glucose transporter gene in Experimental Example 2. In the Figure, “s” indicates that a difference tending to be significant is found at p<0.1, and “**” indicates that it is significant at p<0.01.

FIG. 12 shows the effect of cystine on the glucose absorption capacity during non-exercise in Experimental Example 3.

FIG. 13 shows the effect of combined ingestion of cystine and glutamine on the glucose absorption capacity during exercise in Experimental Example 4. In the Figure, “s” indicates that a difference tending to be significant is found at p<0.1.

### DETAILED DESCRIPTION OF THE PREFERRED EMBODIMENTS

The composition of the present invention for improving decreased absorption in the gastrointestinal tract (hereinafter to be also referred to as “the composition of the present invention” in the present specification”) contains at least one of cystine and glutamine as an active ingredient.

In the present specification, the term “decreased absorption in the gastrointestinal tract” means that hydrolysis of fat, protein, and carbohydrate by intracameral enzymes, digestion with brush border enzymes and uptake of final product, lymphatic transport of nutrient and the like, are disordered for some reason, as a result of which absorption of water, nutrient and the like via the gastrointestinal tract decreases.

In the present specification, the “gastrointestinal tract” is an organ that performs digestion and absorption of food and refers to pharynx, esophagus, stomach, small intestine (duodenum, jejunum, ileum) and large intestine.

In the present specification, the “improvement of decreased absorption” means suppression of the above-mentioned decreased absorption of water, nutrient and the like via the gastrointestinal tract, or improved absorption of water, nutrients and the like via the gastrointestinal tract from a reduced state to a normal or good state.

The cystine contained in the composition of the present invention as an active ingredient, namely, 3,3′-dithiobis(2-aminopropanoic acid), and glutamine, namely, 2-amino-4-carbamoylbutanoic acid, may be used in any of the L-form D-form and DL-form, preferably, the L-form or DL-form, and further preferably, the L-form.

In the present invention, the cystine and glutamine may be used not only in a free form but also a salt form. The terms “cystine” and “glutamine” in the present specification are concepts including even a salt. The salt form includes an acid addition salt, a salt with a base and the like, and a pharmacologically acceptable salt is preferably selected.

Specific examples include salts with an inorganic base, organic base, an inorganic acid or an organic acid, salts with an amino acid and the like.

Examples of the salts with inorganic bases include salts with alkali metals such as lithium, sodium, potassium and the like, salts with alkaline earth metals such as magnesium, calcium and the like, ammonium salt and the like.

Examples of the salts with organic bases include salts with alkanolamine such as monoethanolamine, diethanolamine, triethanolamine and the like, salts with heterocyclic amine such as morpholine, piperidine and the like, and the like.

Examples of the salts with inorganic acids include salts with hydrohalic acid (hydrochloric acid, hydrobromic acid, hydroiodic acid etc.), sulfuric acid, nitric acid, phosphoric acid and the like.

Examples of the salts with organic acids include salts with monocarboxylic acid such as formic acid, acetic acid, propanoic acid and the like; salts with saturated dicarboxylic acid such as oxalic acid, malonic acid, malic acid, succinic acid and the like; salts with unsaturated dicarboxylic acid such as maleic acid, fumaric acid and the like; salts with tricarboxylic acid such as citric acid and the like; salts with keto acid such as α-ketoglutaric acid and the like.

Examples of the salts with amino acid include salts with an aliphatic amino acid such as glycine, alanine and the like; salts with an aromatic amino acid such as phenylalanine and the like; salts with a basic amino acid such as lysine and the like; salts with an acidic amino acid such as aspartic acid, glutamic acid and the like; salts with an amino acid that forms a lactam such as pyroglutamic acid and the like; and the like.

The above-mentioned salts may each be a hydrate (water-containing salt), and examples of such hydrate include 1 hydrate-6 hydrate and the like.

In the present invention, only one of the above-mentioned “cystine” and “glutamine” in a free form or a salt form may be used alone, or two or more kinds thereof may be used in combination.

Each of the “cystine” and “glutamine” is preferably in a free form, in the form of hydrochloride or the like for the object of the present invention.

Cystine and glutamine in a free form or a salt form and used in the present invention may be obtained by extraction and purification from natural animals and plants and the like, or obtained by a chemical synthesis method, a fermentation method, an enzyme method, a gene recombinant method or the like. Commercially available products provided by each company may also be utilized.

The composition of the present invention contains at least one or more kinds of cystine in a free form or a salt form, or one or more kinds of glutamine in a free form or a salt form.

The content of cystine in the composition of the present invention is preferably not less than 0.1 wt %, more preferably 1 wt % to 90 wt %, further preferably 5 wt % to 50 wt %, relative to the total content of amino acid in the composition of the present invention.

The content of glutamine in the composition of the present invention is preferably not less than 0.1 wt %, more preferably 1 wt % to 90 wt %, further preferably 5 wt % to 50 wt %, relative to the total content of amino acid in the composition of the present invention.

In the present specification, the content of each of cystine and glutamine in the composition of the present invention when the amino acid is contained in the form of a salt is shown by the content converted to that of a free form.

As described below, cystine and glutamine each suppress a decrease in the expression, or recover the decreased expression, or promote an increase in the expression, of different digestion and absorption-related genes in the small intestine. Thus, it is preferable to contain both cystine and glutamine in the composition of the present invention.

When the composition of the present invention contains both cystine and glutamine, and the ratio of these contents (cystine:glutamine) is preferably 1:0.01 to 1:100, more preferably 1:0.1 to 1:10, in weight ratio.

The composition of the present invention may further contain, in addition to at least one of cystine and glutamine, other nutrition components such as a carbohydrate, lipid, protein, amino acid other than cystine and glutamine, vitamin, mineral and the like.

The composition of the present invention can be formulated into various forms such as liquids such as a solution, suspension, emulsion and the like; a semi-solid such as a gel, cream and the like; a solid such as a powder, granule, tablet, capsule and the like, and the like by adding, where necessary, other nutrition components and pharmaceutically acceptable additives to at least one of cystine and glutamine and according to a formulating means well known in the field of preparations, for example, the methods described in the Japanese Pharmacopoeia XVII General Rules for preparations [3] Monographs for Preparations and the like, which is incorporated herein by reference in its entirety.

The above-mentioned pharmaceutically acceptable additive can be appropriately selected according to the form of the composition of the present invention and, for example, an excipient, binder, disintegrant, lubricant, coating agent, base, solvent, solubilizing agents, solubilizer, emulsifier, dispersing agent, suspending agent, stabilizer, thickener, soothing agent, isotonicity agent, pH adjuster, antioxidant, antiseptic, preservative, corrigent, sweetening agent, flavor, colorant and the like can be mentioned.

To be specific, examples of the excipient include magnesium carbonate, saccharides (glucose, lactose, cornstarch etc.), sugar alcohol (sorbitol, mannitol etc.) and the like.

Examples of the binder include gelatin, pregelatinized starch, partly pregelatinized starch, cellulose and a derivative thereof (crystalline cellulose, hydroxypropylcellulose etc.) and the like.

Examples of the disintegrant include crospovidone, povidone, crystalline cellulose and the like.

Examples of the lubricant include talc, magnesium stearate and the like.

Examples of the coating agent include methacrylic acid.methyl methacrylate copolymer, methacrylic acid.ethyl acrylate copolymer, methyl methacrylate.butyl methacrylate.methacrylic acid dimethylaminoethyl copolymer, ethyl acrylate.methyl methacrylate.methacrylic acid trimethylammonium chloride ethyl copolymer and the like.

Examples of the base include animal and plant fats and oils (olive oil, cacao butter, beef tallow, sesame oil, hydrogenated oil, castor oil etc.), wax (Carnauba wax, beeswax etc.), polyethylene glycol and the like.

Examples of the solvent include purified water, water for injection, monovalent alcohol (ethanol etc.), polyhydric alcohol (glycerol etc.) and the like.

Examples of the solubilizing agent include propylene glycol, medium-chain triglyceride and the like.

Examples of the solubilizer, emulsifier, dispersing agent and suspending agent include surfactant such as sorbitan fatty acid ester, glycerol fatty acid ester, polyoxyethylene sorbitan fatty acid ester (polysorbate 20, polysorbate 80 etc.), polyoxyethylene hydrogenated castor oil, sucrose fatty acid ester and the like, and the like.

Examples of the stabilizer include adipic acid, β-cyclodextrin, ethylenediamine, sodium edetate and the like.

Examples of the thickener include water-soluble polymer (sodium polyacrylate, carboxyvinyl polymer etc.), polysaccharides (sodium alginate, xanthan gum, tragacanth etc.) and the like.

Examples of the soothing agent include ethyl aminobenzoate, chlorobutanol, propylene glycol, benzyl alcohol and the like.

Examples of the isotonicity agent include potassium chloride, sodium chloride, sorbitol, saline and the like.

Examples of the pH adjuster include hydrochloric acid, sulfuric acid, acetic acid, citric acid, lactic acid, sodium hydroxide, potassium hydroxide and the like.

Examples of the antioxidant include dibutylhydroxytoluene (BHT), butylhydroxyanisole (BHA), dl-α-tocopherol, erythorbic acid and the like.

Examples of the antiseptic and preservative include paraben (methylparaben etc.), benzyl alcohol, sodium dehydroacetate, sorbic acid and the like.

Examples of the corrigent include ascorbic acid, erythritol, L-sodium glutamate and the like.

Examples of the sweetening agent include aspartame, licorice extract, saccharin and the like.

Examples of the flavor include 1-menthol, d-camphor, vanillin and the like.

Examples of the colorant include tar pigment (Food Color Red No. 2, Food Color Blue No. 1, Food Color yellow No. 4 etc.), inorganic pigment (red ferric oxide, yellow iron oxide, black iron oxide etc.), natural dye (turmeric extract, β-carotene, sodium copper-chlorophyllin etc.) and the like.

In the present invention, one or more kinds of the above-mentioned additives can be used.

The daily ingestion amount or dose of the composition of the present invention is appropriately determined according to the kind, sex, age of the target to be applied to (hereinafter to be also referred to as the “application target” in the present specification), condition and level of decreased absorption in the gastrointestinal tract observed in the application target, the form of the composition of the present invention, administration method and the like. When the application target is a human adult, it is generally 0.1 mg/kg of body weight to 5000 mg/kg of body weight, preferably 1 mg/kg of body weight to 2500 mg/kg of body weight, more preferably 10 mg/kg of body weight to 1000 mg/kg of body weight, as the amount of at least one of cystine and glutamine (converted to the amount of a free form) (total amount of cystine and glutamine when they are used in combination (total amount when converted to free form)).

The above-mentioned amount can be ingested or administered at once or in several portions (e.g., 2 to 3 portions) per day.

In addition, the ingestion or dosing period of the composition of the present invention is also appropriately determined according to the condition, level and the like of the decreased absorption in the gastrointestinal tract observed in the application target. When decreased absorption in the gastrointestinal tract is often caused by stress that is routinely applied or by exercise carried out continuously, or the like, it is preferable to use the composition of the present invention for continuous ingestion or administration for a long period of time.

The composition of the present invention can be formulated as a unit package form. In the present specification, the “unit package form” means a form of one or more units with a particular amount (e.g., ingestion amount or dose per one time etc.) as one unit is/are filled in one container or packed in a package. For example, a unit package form with ingestion amount or dose per one time as one unit is referred to as “unit package form for ingestion amount or dose per one time”. A container or package used for the unit package form can be appropriately selected according to the faint and the like of the composition of the present invention. For example, a paper container or bag, plastic container or bag, pouch, aluminum can, steel can, glass bottle, pet bottle, PTP (press through pack) package sheet and the like can be mentioned.

The application target of the composition of the present invention includes, for example, mammals (e.g., human, monkey, mouse, rat, guinea pig, hamster, rabbit, cat, dog, bovine, horse, donkey, swine, sheep, etc.), birds (e.g., duck, chicken, goose, turkey, etc.) and the like.

When the composition of the present invention is applied to an application subject animal (hereinafter to be also simply referred to as “subject animal”) other than human, the ingestion amount or dose of the composition of the present invention can be appropriately set according to the kind, sex, body weight and the like of the subject animal.

The composition of the present invention can well improve decreased absorption of water, nutrient and the like, which is caused by various reasons, in the gastrointestinal tract. Particularly, it is more effective for decreased absorption of water, nutrients and the like, resulting from gastrointestinal tract disorder and the like induced by stress load or exercise.

Examples of the nutrients whose decreased absorption in the gastrointestinal tract can be improved by the composition of the present invention include proteins such as plant-derived protein (soybean protein etc.), animal-derived protein and the like; peptide; amino acids such as essential amino acid (leucine, isoleucine, valine, threonine etc.), non-essential amino acid (glycine, alanine etc.) and the like; carbohydrates such as monosaccharides (glucose, fructose etc.), disaccharides (maltose, sucrose etc.), oligosaccharide (maltotriose etc.), dextran, dextrin, starch and the like; lipids such as simple lipid (acylglycerol etc.), complex lipid (glycerolphospholipid, sphingophospholipid, glycerolglycolipid, sphingoglycolipid etc.), derived lipid (fatty acid, carotenoid, cholesterol etc.) and the like; vitamins such as vitamin A (retinol, retinal, retinoic acid etc.), vitamin B group (vitamin B₁ (thiamine), vitamin B₂ (riboflavin), niacin (nicotinic acid, nicotinamide), vitamin B₆ (pyridoxal, pyridoxamine, pyridoxine), biotin, folic acid, pantothenic acid, vitamin B₁₂ (cyanocobalamin, hydroxocobalamin) etc.), vitamin C (ascorbic acid etc.), vitamin D (cholecalciferol, ergocalciferol etc.), vitamin E (tocopherol, tocotrienol etc.), vitamin K (phylloquinone, menaquinone, menadione etc.) and the like; minerals such as sodium chloride, potassium chloride, calcium chloride, dipotassium phosphate, magnesium sulfate and the like.

As described below, the composition of the present invention can, in small intestine epithelial cells, improve decreased absorption of water and nutrient in the gastrointestinal tract by suppressing a decrease in the gene expression due to stress load or exercise, or recovering the decreased gene expression, or promoting an increase in the gene expression, of transporters involved in the absorption of nutrient (solute carrier transporter group (SLC family), ATP binding cassette transporter (ABC) group (ABC family), glucose transporter (GLUT), sodium-dependent glucose transporter (SGLT)), and aquaporin involved in the absorption of water and mineral.

As regards decreased absorption of vitamins, particularly, decreased absorption of vitamin B group (biotin, folic acid, pantothenic acid etc.) and liposoluble vitamins (vitamin A, vitamin D, vitamin E etc.) in the gastrointestinal tract can be improved by suppressing a decrease in the gene expression due to stress load or exercise, or recovering the decreased gene expression, or promoting an increase in the gene expression, of biotinidase, folic acid transporter, sodium-dependent multivitamin transporter, and a transporter (scavenge receptor class B etc.) involved in the absorption of liposoluble vitamins (vitamin A, vitamin D, vitamin E etc.).

The composition of the present invention can improve decreased absorption of water and nutrients in the gastrointestinal tract, can prevent expression of various symptoms caused by malabsorption of water and nutrient, or can improve the aforementioned symptoms.

Examples of the symptom caused by malabsorption of water include dehydration, heat disorder and the like.

Examples of the symptom caused by malabsorption of nutrients include hypochromic anemia due to malabsorption of iron; macrocytic anemia due to malabsorption of vitamin B₁₂, folic acid; bleeding, peliosis, petechia due to malabsorption of vitamin K and vitamin C; carpopedal spasm due to malabsorption of calcium, magnesium; edema due to malabsorption of protein; glossitis due to malabsorption of vitamin B₂ and B₁₂, folic acid, niacin, iron; nyctalopia due to malabsorption of vitamin A; pain in the limbs and bones, pathological bone fracture due to malabsorption of potassium, magnesium, calcium, vitamin D; peripheral nerve disorders due to malabsorption of vitamin B₁, B₆, B₁₂ and the like.

Therefore, the composition of the present invention is preferably used for ingestion by or administration to those showing decreased absorption of water, nutrient and the like in the gastrointestinal tract.

As mentioned above, since the composition of the present invention is effective for decreased absorption of water, nutrients and the like in the gastrointestinal tract, which is caused by stress or exercise, it can be more preferably used for ingestion by or administration to those showing decreased absorption of water, nutrients and the like in the gastrointestinal tract due to stress load or exercise.

Thus, the composition of the present invention can be more preferably used for ingestion by or administration to those exposed to constant stress, those in need of daily exercise (e.g., patients under exercise therapy etc.), athletes who routinely perform intense exercises and the like, with the aim to improve decreased absorption of water, nutrient and the like in the gastrointestinal tract.

For ingestion by or administration to those in need of daily exercise, athletes and the like of the composition of the present invention, the composition of the present invention may be given for ingestion or administration before exercise, during exercise or after exercise.

The composition of the present invention can be provided as a pharmaceutical composition (hereinafter to be also referred to as “the pharmaceutical composition of the present invention” in the present specification).

The pharmaceutical composition of the present invention can be formulated into, directly or after further adding the above-mentioned pharmaceutically acceptable additives, a dosage form of oral preparation such as tablet, coating tablet, chewable tablet, pill, (micro)capsule, granule, fine granule, powder, elixir, lemonade, syrup, suspension, emulsion, oral jelly and the like, injection such as solution, suspension, emulsion and the like, solid injection to be used by dissolving or suspending when in use, injectable preparation such as transfusion, sustainable injection and the like, tubal liquid, and the like.

The pharmaceutical composition of the present invention can be preferably administered to patients showing decreased absorption of water, nutrients and the like in the gastrointestinal tract and having symptoms due to malabsorption of water, nutrient and the like, patients showing decreased absorption of water, nutrient and the like in the gastrointestinal tract and feared to express symptoms due to malabsorption of water, nutrient and the like, and the like.

In addition, the pharmaceutical composition of the present invention can be more preferably administered to patients showing decreased absorption of water, nutrients and the like in the gastrointestinal tract due to a gastrointestinal tract disorder and the like caused by stress, patients under continuous exercise therapy and showing decreased absorption of water, nutrients and the like in the gastrointestinal tract due to the exercise, and those suffering from decreased absorption of water, nutrients and the like in the gastrointestinal tract due to the intense exercise.

The pharmaceutical composition of the present invention is administered to the above-mentioned application target such that the daily dose of at least one of cystine and glutamine is the above-mentioned dose per day.

Furthermore, the composition of the present invention can be ingested by adding to various foods. The food to which the composition of the present invention is added is not particularly limited, and may be any as long as it is a food in the form generally served for meals or dessert.

For example, the composition of the present invention is added to drinks such as beverage water and the like, and a suitable flavor is added when desired, whereby a drink can be provided.

More specifically, the composition of the present invention can be added, for example, to beverage water such as fruit juice drinks, sport drinks and the like; dairy products such as milk, yogurt and the like; confectionery such as jelly, chocolate, candy and the like, and the like.

The composition of the present invention is preferably added to the above-mentioned various foods in amounts to be ingested per day such that the ingestion amount of at least one of cystine and glutamine is the above-mentioned dose per day.

In addition, the composition of the present invention can be provided as a food composition (hereinafter to be also referred to as “the food composition of the present invention” in the present specification).

The food composition of the present invention can be prepared as various forms such as liquid, suspension, emulsion, gel, cream, powder, granule, sheet, capsule, tablet and the like directly or by adding general food additives as necessary and according to a general food production technique.

Furthermore, the food composition of the present invention can be prepared as various food forms such as beverage water (fruit juice drinks, sport drinks, coffee drinks, tea drinks etc.), dairy product (lactic fermenting beverage, fermented milk, butter, cheese, yogurt, processed milk, defatted milk etc.), meat product (ham, sausage, hamburger etc.), seafood paste product (fish cake, tube-shaped fish sausage, deep-fried ball of fish paste etc.), egg product (rolled Japanese-style omelette, steamed egg custard etc.), confectionery (cookie, jelly, chewing gum, candy, snack food, frozen dessert etc.), bread, noodles, pickle, dried fish, food boiled in soy sauce, soup, seasoning and the like by adding the composition of the present invention to various food starting materials and adding general food additives as necessary. It may also be a bottled food, canned food or retort pouch food.

As the above-mentioned food additive, a manufacturing agent (brine, binding agent etc.), thickening stabilizer (xanthan gum, sodium carboxymethylcellulose etc.), gelling agent (gelatin, agar, carrageenan etc.), gum base (vinyl acetate resin, jelutong, chicle etc.), emulsifier (glycerol fatty acid ester, sucrose fatty acid ester, saponin, lecithin etc.), preservative (benzoic acid, sodium benzoate, sorbic acid, potassium sorbate, ε-polylysine etc.), antioxidant (ascorbic acid, erythorbic acid, catechin etc.), glazing agent (shellac, paraffin wax, beeswax etc.), fungicide (thiabendazole, fludioxonil etc.), leavening agent (sodium hydrogen carbonate, glucono δ-lactone, alum etc.), sweetener (aspartame, acesulfame potassium, licorice extract etc.), bittering agent (caffeine, naringin, worm wood extract etc.), acidulant (citric acid, tartaric acid, lactic acid etc.), seasoning (L-sodium glutamate, disodium 5′-inosinate etc.), colorant (annatto dye, turmeric dye, gardenia dye etc.), flavor (synthetic flavor such as ethyl acetoacetate, anisealdehyde and the like, natural flavor such as orange, lavender and the like) and the like can be mentioned.

In the present invention, one or more kinds of the above-mentioned food additives can be used.

The food composition of the present invention can be preferably used for ingestion by those showing decreased absorption of water, nutrients and the like in the gastrointestinal tract, or those at risk to show decreased absorption of water, nutrients and the like in the gastrointestinal tract.

In addition, the food composition of the present invention can be more preferably used for ingestion by those exposed to constant stress and at risk to show decreased absorption of water, nutrients and the like in the gastrointestinal tract, or those at risk to show decreased absorption of water, nutrients and the like in the gastrointestinal tract due to exercise such as those receiving an exercise therapy, athletes and the like.

Therefore, the food of the present invention can also be provided as a food with health claims (food for specified health uses, food with nutrient function claims, food with functional claims etc.), food for special dietary uses (food for sick people, food for elderly people etc.), health supplement, dietary supplement or the like for improving decreased absorption of water, nutrients and the like in the gastrointestinal tract.

The food composition of the present invention is preferably ingested by the above-mentioned application target such that the ingestion amount of at least one of cystine and glutamine is the above-mentioned ingestion amount per day.

The above-mentioned composition of the present invention not only improves decreased absorption in the gastrointestinal tract but also shows an effect of promotion of absorption in the gastrointestinal tract.

Therefore, the composition of the present invention also functions as a composition for promoting absorption in the gastrointestinal tract. As used herein, the “promoting absorption in the gastrointestinal tract” refers to absorption of nutrients and the like in the gastrointestinal tract which is improved more than usual.

Therefore, the present invention also provides a composition for promoting absorption in the gastrointestinal tract, which contains at least one of cystine and glutamine as an active ingredient.

The composition for promoting absorption in the gastrointestinal tract of the present invention can be provided as a pharmaceutical composition or food composition, or can also be ingested by adding to food.

In the composition for promoting absorption in the gastrointestinal tract of the present invention, the daily ingestion amount or dose, frequency of daily ingestion or administration, ingestion or dosing period and the like of at least one of cystine and glutamine are the same as those in the above-mentioned composition for improving decreased absorption m in the gastrointestinal tract.

The composition for promoting absorption in the gastrointestinal tract of the present invention can be preferably used for ingestion by or administration to those who need more nutrients, such as those who are engaged in heavy work, athletes who routinely perform intense exercises, growing children, adolescents and the like.

Furthermore, the present invention also provides a method for improving decreased absorption in the gastrointestinal tract of a target animal in need of improvement of decreased absorption in the gastrointestinal tract (hereinafter to be also referred to as “the method of the present invention” in the present specification).

The method of the present invention includes ingestion by or administration to a target in need of improvement of decreased absorption in the gastrointestinal tract of at least one of cystine and glutamine in an amount effective for improving the decreased absorption in the gastrointestinal tract.

The subject animal in the method of the present invention includes mammals (e.g., human, monkey, mouse, rat, guinea pig, hamster, rabbit, cat, dog, bovine, horse, donkey, swine, sheep etc.), birds (e.g., duck, chicken, goose, turkey etc.) and the like.

The method of the present invention is effective for improving decreased absorption in the gastrointestinal tract caused by various reasons, and is more effective for improving decreased absorption in the gastrointestinal tract caused by stress or exercise.

In the case of human, the method of the present invention is preferably applied to patients showing decreased absorption in the gastrointestinal tract, particularly, those showing decreased absorption in the gastrointestinal tract resulting from a gastrointestinal tract disorder and the like due to stress, those exposed to constant stress and at risk to show decreased absorption in the gastrointestinal tract, or patients receiving an exercise therapy or athletes showing decreased absorption in the gastrointestinal tract and at risk to show decreased absorption in the gastrointestinal tract, and the like.

While the effective amount of at least one of cystine and glutamine in the method of the present invention is determined according to the kind, age, sex, the condition, level and the like of the decreased absorption in the gastrointestinal tract of the application target, an amount similar to the above-mentioned ingestion amount or dose of the composition of the present invention for a human or a subject animal other than human can be ingested or administered at the frequency and in the period mentioned above.

The ingestion or administration method of at least one of cystine and glutamine in the method of the present invention includes oral administration, enteral tube administration, administration by infusion and the like. Oral administration is preferable since convenient ingestion is possible without the need to be performed under the guidance and supervision of a doctor at a medical institution.

As described above, at least one of cystine and glutamine not only improves decreased absorption in the gastrointestinal tract but also has an effect of promotion of absorption in the gastrointestinal tract. Therefore, the present invention also provides a method for promoting absorption in the gastrointestinal tract, including ingestion by or administration to a target animal in need of promotion of absorption in the gastrointestinal tract of at least one of cystine and glutamine in an amount effective for promoting the absorption in the gastrointestinal tract of the target animal.

The effective amount, frequency and period of ingestion or administration of at least one of cystine and glutamine are the same as those in the method for improving decreased absorption in the gastrointestinal tract.

The method for promoting absorption in the gastrointestinal tract of the present invention can be preferably applied to those who need more nutrients, such as those who are engaged in heavy work, athletes who routinely perform intense exercises, growing children, adolescents and the like.

Other features of the invention will become apparent in the course of the following descriptions of exemplary embodiments which are given for illustration of the invention and are not intended to be limiting thereof.

### Examples

### Experimental Example 1. Study of Effect of Cystine and Glutamine on Expression of Digestion Absorption Related Gene in Small Intestine

7-Week-old male CD2F1 mice (CHARLES RIVER LABORATORIES JAPAN, INC.) were divided into 4 groups (“Sed”, “Ex”, “Ex+Gln” and “Ex+Cys2”) (n=6/group), Sed group and Ex group were made to ingest a standard purified feed (AIN-93G composition), Ex+Gln group was made to ingest a standard purified feed (AIN-93G composition) added with glutamine (2 wt %) by replacing casein, and Ex+Cys2 group was made to ingest a standard purified feed (AIN-93G composition) added with cystine (2 wt %) by replacing casein, each for 7 days. Thereafter, each group was fasted overnight, and Ex group, Ex+Gln group and Ex+Cys2 group were made to run in a running wheel for 4 hr (speed=10.5 m/min). Sed group was continuously fasted during the running exercise of Ex group, Ex+Gln group and Ex+Cys2 group. At 1 hr after completion of running, the small intestine was collected from the mice of each group, and total RNA was extracted using Rneasy Lipid Tissue Mini Kit (QIAGEN). GeneChip Mouse Genome 430 2.0 (3′IVT) array (Affymetrix) was used for the measurement of comprehensive gene expression. Difference in the gene expression level of each group was tested by Dunnett's test following one-way analysis of variance.

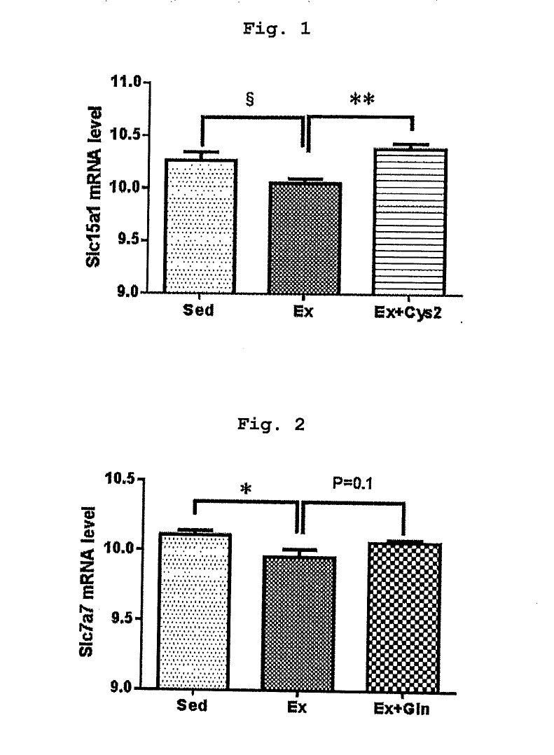

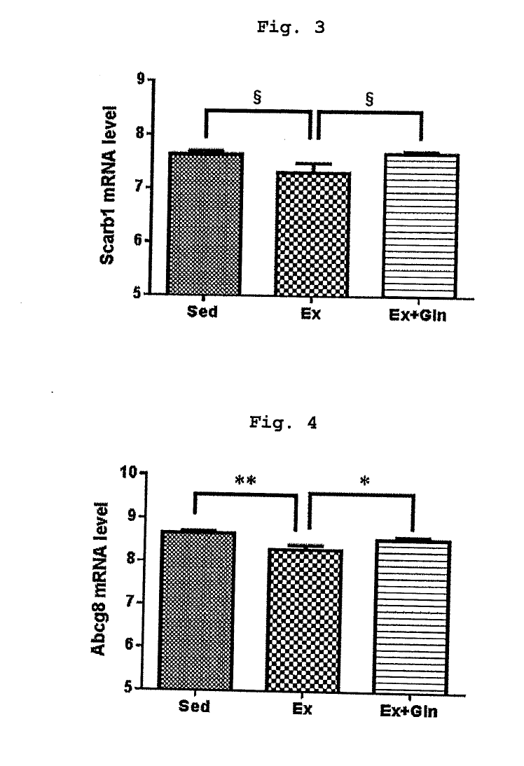

The expression levels of genes (Slc15a1, Slc7a7, Scarb1, Abcg8, Abcg5, Slc46a1, Btd, Slc5a6 and Aqa3) respectively encoding peptide transporter involved in the absorption of proteins and peptides, amino acid transporter involved in the absorption of amino acids, scavenge receptor class B involved in the absorption of lipids and liposoluble vitamins, ABC protein G8 and ABC protein G5 involved in the absorption of cholesterol, folic acid transporter involved in the absorption of folic acid, biotinidase involved in the absorption of biotin, sodium-dependent multivitamin transporter involved in the absorption of pantothenic acid, biotin and lipoic acid, and aquaporin involved in the absorption of water and mineral are shown in FIGS. 1 to 9 for each mRNA level. The measurement results of the amount of mRNA in each group are shown as the mean value±standard error of the mean value of 6 mice.

As shown in FIG. 1 to FIG. 9, the expression level of each of the above-mentioned genes decreased in the group (Ex) given the standard purified feed with exercise as compared to the group (Sed) given the standard purified feed without exercise (peptide transporter (tendency toward significance at P<0.1 for each gene of peptide transporter, scavenge receptor class B, significant at P<0.05 for each gene of amino acid transporter, folic acid transporter, significant at P<0.01 for each gene of ABC protein G8, ABC protein G5, biotinidase, sodium-dependent multivitamin transporter, aquaporin). From the results, it was confirmed that the absorption of protein, peptide, amino acid, lipid, vitamin, mineral and water decreased due to the running exercise for 4 hr.

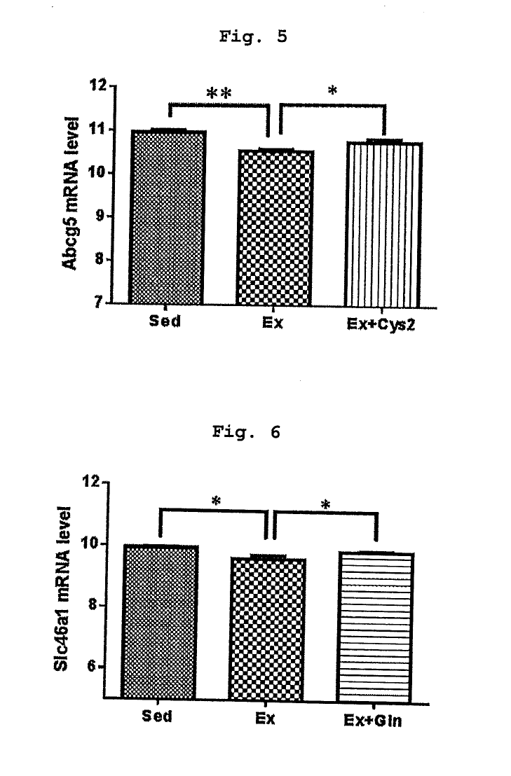

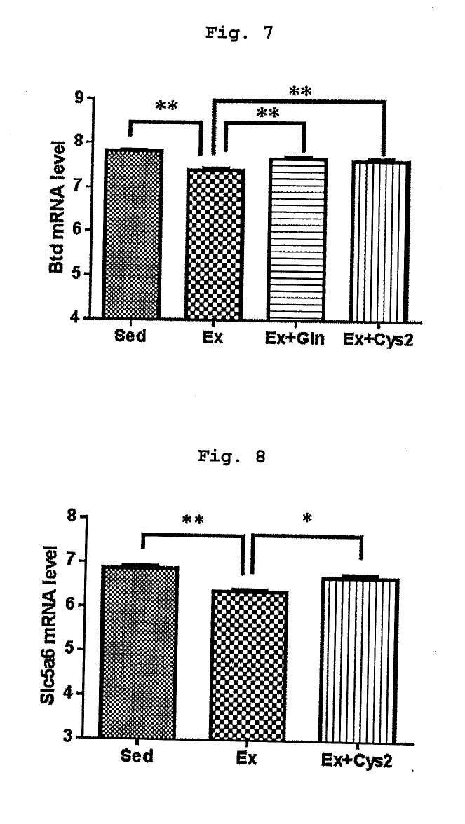

On the other hand, as shown in FIG. 1, FIG. 5, and FIG. 7 to FIG. 9, the expression level of each gene of peptide transporter, ABC protein G5, biotinidase, sodium-dependent multivitamin transporter and aquaporin in the group (Ex+Cys2) given the standard purified feed added with cystine with exercise increased as compared to the group (Ex) given the standard purified feed with exercise (significant at P<0.01 for each gene of peptide transporter, biotinidase, aquaporin, significant at P<0.05 for ABC protein G5, sodium-dependent multivitamin transporter).

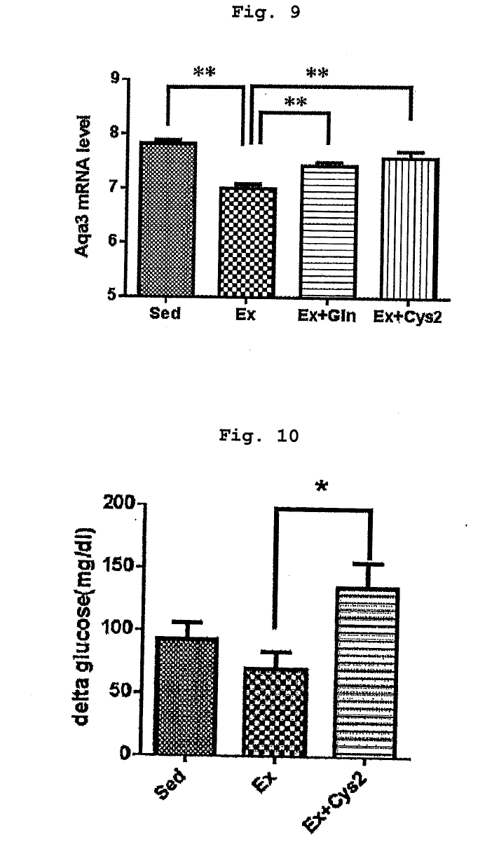

As shown in FIG. 2 to FIG. 4, FIG. 6, FIG. 7, and FIG. 9, the expression level of each gene of amino acid transporter, scavenge receptor class B, ABC protein G8, folic acid transporter, biotinidase and aquaporin increased in the group (Ex+Gln) given the standard purified feed added with glutamine with exercise, as compared to the group (Ex) given the standard purified feed with exercise (tendency toward significance at P=0.1 for amino acid transporter gene, tendency toward significance at P<0.1 for scavenge receptor class B gene, significant at P<0.05 for each gene of ABC protein G8, folic acid transporter, significant at P<0.01 for each gene of biotinidase, aquaporin).

From the above-mentioned results, it was shown that cystine and glutamine improve decreased absorption of water and nutrient due to exercise. It was also found that there are different genes related to digestion and/or absorption in the small intestine that glutamine or cystine suppresses or recovers a decrease in the expression, or promotes an increase in the expression.

It was suggested therefore that combined use of cystine and glutamine is preferable for effectively improving decreased absorption of water and nutrient due to exercise.

### Experimental Example 2. Study of Effect of Exercise and Cystine on Carbohydrates Absorption in Small Intestine and Sodium-Dependent Glucose Transporter Gene Expression

7-Week-old male CD2F1 mice (CHARLES RIVER LABORATORIES JAPAN, INC.) were divided into 3 groups (“Sed”, “Ex” and “Ex+Cys2”) (n=12/group), Sed group and Ex group were made to ingest a standard purified feed (AIN-93G composition), and Ex+Cys2 group was made to ingest a standard purified feed (AIN-93G composition) added with cystine (2 wt %) by replacing casein, each for 7 days. Thereafter, each group was fasted overnight, and Ex group and Ex+Cys2 group were made to run in a running wheel for 4 hr (speed=10.5 m/min). Sed group was continuously fasted during the running exercise of Ex group and Ex+Cys2 group. Immediately after completion of running, the small intestine was collected, everted intestine samples were produced according to the method of KirK et al. (ADVANCES In Physiology Education 37 (4) 415-426 (2013), which is incorporated herein by reference in its entirety). As the everted intestine sample, a part of 8 cm below the point of 4 cm from the pyloric part was used.

Ringer buffer containing 10 mM glucose was added to the serous membrane side and the villus side of the produced everted intestine sample of each group, and the sample was incubated under oxygen supply at 37° C. for 90 min. Thereafter, the everted intestine sample was taken out, and the glucose concentration of the Ringer buffer on the serous membrane side and the villus side was measured using a glucose measurement kit (“glucose CII Test Wako” (Wako Pure Chemical Industries, Ltd.)). The glucose absorption capacity of each group was calculated from the difference in the glucose concentration between the villus side and the serous membrane side.

Furthermore, using Rneasy Lipid Tissue Mini Kit (QIAGEN), total RNA was extracted from a part of the small intestine used for producing the everted intestine sample. QuantStudio 12K Flex Real-Time PCR System (Thermo Fisher Scientific) was used for the measurement of the expression of sodium-dependent glucose transporter gene (SGLT1). Difference in the glucose absorption capacity and the gene expression level of each group were tested by Dunnett's test following one-way analysis of variance.

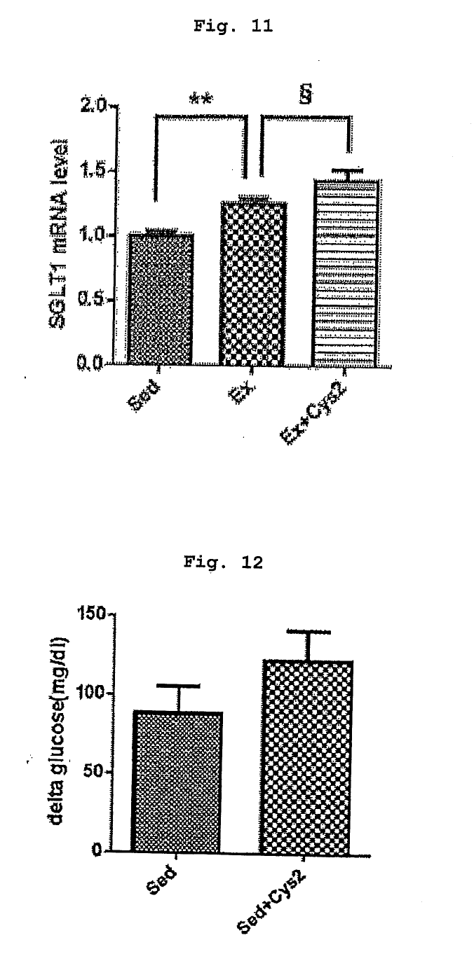

The glucose absorption capacity of each group is shown in FIG. 10 as the difference in the glucose concentration between the villus side and the serous membrane side. In addition, the expression level of the sodium-dependent glucose transporter gene (SGLT1) is shown in FIG. 11 as the mRNA amount. These are shown as the mean value±standard error of the mean value of 12 mice.

As shown in FIG. 10, the glucose absorption capacity decreased in the group (Ex) given the standard purified feed with exercise as compared to the group (Sed) given the standard purified feed without exercise.

On the other hand, the glucose absorption capacity was significantly (P<0.05) improved in the group (Ex+Cys2) given the standard purified feed added with cystine with exercise increased as compared to the group (Ex) given the standard purified feed with exercise.

Furthermore, it was found that cystine ingestion improved not only a decrease in the glucose absorption capacity due to the exercise but also the glucose absorption capacity, since the glucose absorption capacity in Ex+Cys2 was higher than that in the Sed group.

As shown in FIG. 11, expression of the gene (SGLT1) of sodium-dependent glucose transporter involved in glucose absorption increased in the group (Ex) given the standard purified feed with exercise as compared to the group (Sed) given the standard purified feed without exercise (peptide transporter (significant at P<0.01). Furthermore, the expression of SGLT1 tended to increase in the group (Ex+Cys2) given the standard purified feed added with cystine with exercise, as compared to the group (Ex) given the standard purified feed with exercise (tendency toward significance at P<0.1).

From the above-mentioned results of Experimental Example 2, it was confirmed that the glucose absorption capacity decreases due to the running exercise for 4 hr, but cystine ingestion improves the decrease and cystine ingestion further enhances the glucose absorption capacity more than the general level.

It was also confirmed that cystine increases expression of sodium-dependent glucose transporter (SGLT1) gene more than usual.

### Experimental Example 3. Study of Effect of Cystine on Carbohydrate Absorption in Small Intestine without Exercise

7-Week-old male CD2F1 mice (CHARLES RIVER LABORATORIES JAPAN, INC.) were divided into 2 groups (“Sed” and “Sed+Cys2”) (n=6/group), Sed group was made to ingest a standard purified feed (AIN-93G composition), and Sed+Cys2 group was made to ingest a standard purified feed (AIN-93G composition) added with cystine (2 wt %) by replacing casein, each for 7 days. Then, each group was fasted overnight, and the small intestine was collected, everted intestine samples were produced according to the method of KirK et al. (ADVANCES In Physiology Education 37 (4) 415-426 (2013), which is incorporated herein by reference in its entirety). As the everted intestine sample, a part of 8 cm below the point of 4 cm from the pyloric part was used.

Ringer buffer containing 10 mM glucose was added to the serous membrane side and the villus side of the everted intestine sample of each group, and the sample was incubated under oxygen supply at 37° C. for 90 min. Thereafter, the everted intestine sample was taken out, and the glucose concentration of the Ringer buffer on the serous membrane side and the villus side was measured using a glucose measurement kit (“glucose CII Test Wako” (Wako Pure Chemical Industries, Ltd.)). The glucose absorption capacity of each group was calculated from the difference in the glucose concentration between the villus side and the serous membrane side. The difference in the glucose absorption capacity of each group was determined by a comparison t-test between two groups.

The glucose absorption capacity of each group is shown in FIG. 12 as the difference in the glucose concentration between the villus side and the serous membrane side. The measurement results of the difference in the glucose concentration shown as the mean value±standard error of the mean value of 6 mice.

As shown in FIG. 12, the glucose absorption capacity was improved in the group (Sed+Cys2) given the standard purified feed added with cystine increased as compared to the group (Sed) given the standard purified feed.

From the above-mentioned results of Experimental Example 3, it was confirmed that, during non-exercise, cystine ingestion improves glucose absorption capacity and promotes glucose absorption.

### Experimental Example 4. Study of Combined Ingestion of Cystine and Glutamine on Carbohydrate Absorption in Small Intestine During Exercise

7-Week-old male CD2F1 mice (CHARLES RIVER LABORATORIES JAPAN, INC.) were divided into 3 groups (“Sed”, “Ex+CG1” and “Ex+CG2”) (n=6-8/group), Ex group was made to ingest a standard purified feed (AIN-93G composition), Ex+CG1 group was made to ingest a standard purified feed (AIN-93G composition) added with cystine (0.6 wt %) and glutamine (2.0 wt %) by replacing casein, and Ex+CG2 group was made to ingest a standard purified feed (AIN-93G composition) added with cystine (2.0 wt %) and glutamine (2.0 wt %) by replacing casein, each for 7 days.

Thereafter, each group was fasted overnight and made to run in a running wheel for 4 hr (speed=10.5 m/min). Immediately after completion of running, the small intestine was collected and everted intestine samples were produced according to the method of KirK et al. (ADVANCES In Physiology Education 37 (4) 415-426 (2013), which is incorporated herein by reference in its entirety). As the everted intestine sample, a part of 8 cm below the point of 4 cm from the pyloric part was used.

Ringer buffer containing 10 mM glucose was added to the serous membrane side and the villus side of the produced everted intestine sample of each group, and the sample was incubated under oxygen supply at 37° C. for 90 min. Thereafter, the everted intestine sample was taken out, and the glucose concentration of the Ringer buffer on the serous membrane side and the villus side was measured using a glucose measurement kit (“glucose CII Test Wako” (Wako Pure Chemical Industries, Ltd.)). The glucose absorption capacity of each group was calculated from the difference in the glucose concentration between the villus side and the serous membrane side.

The difference in the glucose absorption capacity of each group was tested by Dunnett's test following one-way analysis of variance.

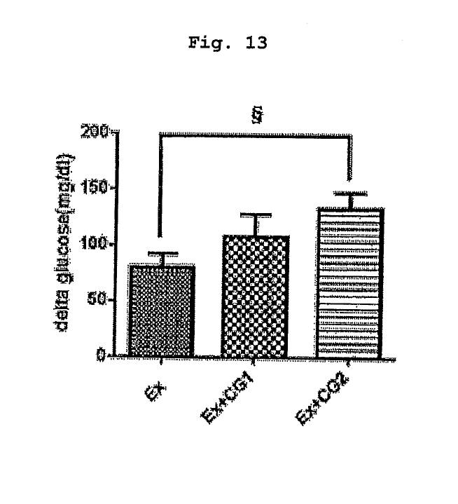

The glucose absorption capacity of each group is shown in FIG. 13 as the difference in the glucose concentration between the villus side and the serous membrane side. The measurement results of the difference in the glucose concentration shown as the mean value±standard error of the mean value of 6-8 mice.

As shown in FIG. 13, the glucose absorption capacity tended to increase in the groups (Ex+CG1 and Ex+CG2) given the standard purified feed added with cystine and glutamine increased as compared to the group (Ex) given the standard purified feed with exercise (tendency toward significance at P<0.1 between Ex and Ex+CG2).

From the above-mentioned results of Experimental Example 4, it was confirmed that, during exercise, combined ingestion of cystine and glutamine improves glucose absorption capacity and promotes glucose absorption.

### Example 1. Composition for Improving Decreased Absorption in Gastrointestinal Tract

Cystine and glutamine were mixed at a weight ratio of 7:30 to give the preparation of Example 1.

### INDUSTRIAL APPLICABILITY

As described in detail above, the present invention can provide a composition for improving decreased absorption in the gastrointestinal tract, which can finely improve decreased absorption of water, nutrient and the like in the gastrointestinal tract which is caused by various reasons.

The composition of the present invention for improving decreased absorption in the gastrointestinal tract can suppress decreased absorption via the gastrointestinal tract, and can improve absorption via the gastrointestinal tract from a decreased state to a normal state or a good state.

The composition of the present invention for improving decreased absorption in the gastrointestinal tract is particularly effective for decreased absorption in the gastrointestinal tract which is due to stress or exercise.

In addition, the present invention can provide a composition for promoting absorption in the gastrointestinal tract, which can promote absorption of nutrient and the like in the gastrointestinal tract.

The composition of the present invention for promoting absorption in the gastrointestinal tract can improve efficiency of utilization of nutrient and the like.

Where a numerical limit or range is stated herein, the endpoints are included. Also, all values and subranges within a numerical limit or range are specifically included as if explicitly written out.

As used herein the words “a” and “an” and the like carry the meaning of “one or more.”

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A composition for improving decreased absorption in the gastrointestinal tract, comprising at least one of cystine and glutamine as an active ingredient.

2. The composition according to claim 1, comprising cystine and glutamine.

3. The composition according to claim 2, wherein a weight ratio of cystine to glutamine (cystine:glutamine) is 1:0.01 to 1:100.

4. The composition according to claim 1, which is a pharmaceutical composition.

5. The composition according to claim 1, which is a food composition.

6. A method for improving decreased absorption in the gastrointestinal tract, comprising administering to a subject in need thereof at least one of cystine and glutamine.

7. The method according to claim 6, comprising administering cystine and glutamine.

8. The method according to claim 7, wherein said cystine and glutamine are administered in a weight ratio of cystine to glutamine (cystine:glutamine) of 1:0.01 to 1:100.

9. The method according to claim 6, which improves decreased absorption of water in the gastrointestinal tract.

10. The method according to claim 6, which improves decreased absorption of a nutrient in the gastrointestinal tract.

11. The method according to claim 10, wherein said nutrient is at least one member selected from the group consisting of a protein, a peptide, an amino acid, a carbohydrate, a lipid, a vitamin, and a mineral.

12. The method according to claim 11, wherein said vitamin is at least one member selected from the group consisting of vitamin A, a vitamin B group vitamin, vitamin D, and vitamin E.

13. A method for promoting absorption in the gastrointestinal tract, comprising administering to a subject in need thereof at least one of cystine and glutamine.

14. The method according to claim 13, comprising administering cystine and glutamine.

15. The method according to claim 14, wherein said cystine and glutamine are administered in a weight ratio of cystine to glutamine (cystine:glutamine) of 1:0.01 to 1:100.

16. The method according to claim 13, which promotes absorption of a nutrient in the gastrointestinal tract.

17. The method according to claim 16, wherein said nutrient is at least one member selected from the group consisting of a protein, a peptide, an amino acid, a carbohydrate, a lipid, a vitamin, and a mineral.
